(19) United States
(12) Patent Application Publication
(10) Pub. No.: US 2010/0172854 A1
(43) Pub. Date: Jul. 8, 2010

(54) ANTIBACTERIAL FORMULATIONS

(76) Inventors: Scott Seville, West Yorkshire (GB); Hazel Anne Wilkinson, South Yorkshire (GB)

Correspondence Address: BELL & ASSOCIATES, 58 West Portal Avenue No. 121, SAN FRANCISCO, CA, 94127, US

(21) Appl. No.: 12/531,715

(22) PCT Filed: Mar. 14, 2008
(86) PCT No.: PCT/GB2008/000901

§ 371 Date: Jan. 18, 2010

(30) Foreign Application Priority Data
Mar. 17, 2007 (GB) 0705179.0

(51) Int. Cl. A61K 8/34; A61K 31/05; A61Q 15/00; A61P 31/04

(52) U.S. Cl. 424/65; 514/734

(57) ABSTRACT

Use of a compound of formula (III) below: wherein each of R¹ and R² is independently selected from methyl and ethyl and R³ is hydrogen, or a derivative thereof, as an antibacterial agent. The compound (III) or derivative may be used for pharmaceutical purposes, in particular for the treatment of acne, staphylococcal infections or body odour, or for non-pharmaceutical purposes such as for the disinfection of inanimate surfaces.

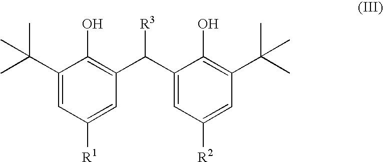

### FIELD OF THE INVENTION

This invention relates to the use of certain compounds as antibacterial agents.

### BACKGROUND TO THE INVENTION

The compound known as “Antioxidant 2246” or “AO 2246”, the chemical names for which include 2,2′-methylene-bis(4-methyl-6-tert-butylphenol); bis(2-hydroxy-3-tert-butyl-5-methylphenyl)methane; 6,6′-di-tert-butyl-2,2′-methylene-di-p-cresol; methane-2,2′-bis(6-t-butyl-p-cresol)-phenol; and 2,2′-methylene-bis(6-(1,1-dimethylethyl)-4-methyl-p-cresol, has the formula (I) shown below.

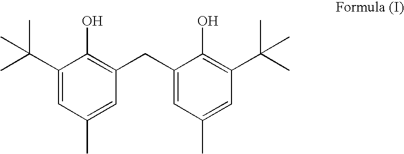

It is known to act as an antioxidant, and has been used as a preservative in for example plastics and elastomeric materials and cosmetics. In the polymer industry it is used as a thermostabiliser, an antioxidant and a free radical polymerisation inhibitor, for example in plastics films intended for the wrapping of foodstuffs. In U.S. Pat. No. 4,891,391 it is disclosed, as one of several possible antioxidants, for use in stabilising antimicrobial actives in thermoplastic compositions, in particular to improve resistance to degradation of antimicrobial activity on heating. In U.S. Pat. No. 5,070,105 it is mentioned for use as a stabiliser in antimicrobial compositions containing the active 2,2-dibromo-3-nitrilopropionamide, the compositions being intended in particular for use as slimicides in paper pulping processes and cooling towers and as sterilising agents for dry cleaning fluids. JP-05-271018 refers to its use as a stabiliser in an insect repellant composition containing empenthrin as the active ingredient.

Another structurally similar compound, also used as an antioxidant and stabiliser, has the formula (II) below:

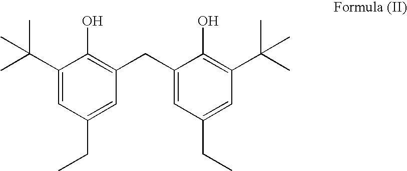

and is commonly known as “Antioxidant 425” or “AO 425”, or as 2,2′-methylene-bis(4-ethyl-6-tert-butylphenol).

Bis-phenol compounds which are structurally similar to Antioxidant 2246 and Antioxidant 425 have also been described for use in modulating cellular immune reactions (WO-96/40093), and for the treatment of hyperlipidemia and arteriosclerosis (EP-0 583 665 and U.S. Pat. No. 3,873,726). JP-6080563 discloses the use of Antioxidant 2246 to lower blood cholesterol levels and suppress macrophage foaming, whilst Pliss et al (Chem. Abs. No. 82:38563) and Choi et al (Chem. Abs. No. 125:237608) have described its anti-tumour activity.

In U.S. Pat. No. 4,985,465 various bis-phenol derivatives, including the sterically hindered bis-di-t-butylphenols, are described for use as antiviral agents. U.S. Pat. No. 6,855,341 discloses an antiviral composition containing a zinc compound and a phenolic antioxidant, which may be a substituted methylene-bis-phenol such as AO 2246.

Kawada et al in J. Antibact. Antifung. Agents, Vol. 9, No. 9: 429-433 describe the use of certain alkylphenol dimers and oligomers as antimicrobial agents. They teach that in an alkyl-substituted phenol, alkyl chain length and molecular weight appear capable of affecting antimicrobial activity. Kawada tested a range of structurally related dimers and oligomers, although not Antioxidants 2246 or 425, against a number of bacterial and fungal strains. His test data showed significant variations in activity between the different test compounds. For example, di-t-butyl-substituted compounds showed a lower level of activity against S. aureus than their di-n-butyl- or di-hexyl-substituted analogues. The data also showed reduced performance against S. aureus for di-alkyl-substituted bis-phenol dimers than for their monomer counterparts, many of the dimers having relatively low activity. For all of the test compounds, activity levels varied significantly depending on the micro-organism used. Thus overall Kawada's findings suggest that alkyl-substituted bis-phenol compounds might not be reliable candidates for antimicrobial uses, and in particular that the t-butyl-substituted dimers would not be expected to perform particularly well in this context.

Kim et al in J. Antibiot., 1995, 49: 31-36 describe the use of a compound they believe to be Antioxidant 2246, isolated from the fungus Aspergillus fumigatus, as an inhibitor of the enzyme acyl-CoA: cholesterol acyltransferase (ACAT). They also report that the compound they isolated has anti-tumour activity, but no in vitro antimicrobial activity against a range of test bacteria.

It has now surprisingly been found that Antioxidant 2246 and certain related antioxidant compounds can be antibacterially active, in particular against both aerobic and anaerobic Gram-positive bacteria and also against selected anaerobic Gram-negative bacteria, and that they can therefore be used as antibacterial agents.

### STATEMENTS OF THE INVENTION

According to a first aspect of the present invention there is provided the use of a compound of formula (III):

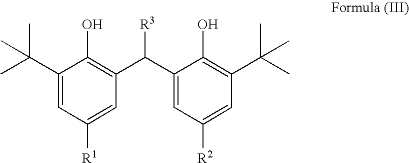

wherein each of R¹ and R² is independently selected from methyl and ethyl and R³ is hydrogen, or a derivative thereof, as an antibacterial agent.

Use as an antibacterial agent embraces use against a condition affecting the human or animal body, which condition is caused, transmitted and/or exacerbated by (in particular caused or transmitted by) bacterial activity.

Thus a second aspect of the present invention provides a compound of formula (III), or a pharmaceutically acceptable derivative thereof, for use in the treatment of a condition affecting the human or animal body, which condition is caused by, transmitted by and/or exacerbated by (in particular either caused or transmitted by) bacterial activity. Such a condition may be for example a skin, skin structure, oral, ocular, aural, nasal, soft tissue or vaginal condition.

In the context of the present invention, treatment of a condition encompasses both therapeutic and prophylactic treatment, of either an infectious or a non-infectious condition, in either a human or animal but in particular a human. It may involve complete or partial eradication of the condition, removal or amelioration of associated symptoms, arresting subsequent development of the condition, and/or prevention of, or reduction of risk of, subsequent occurrence of the condition. It will typically involve use of the compound (III) or derivative as a bactericide. It may involve the prophylactic treatment of any area of the body, in particular the skin or nares or another epithelial or mucosal surface, against bacterial infections, including against staphylococcal infections such as those associated with MRSA.

The treatment of a condition also embraces the prevention, or reduction of risk of, dissemination or transmission of the condition, for example person to person. In this context, the compound (III) or derivative may be used as a disinfectant against the relevant micro-organism, for example for antisepsis of the skin and/or other appropriate parts of the body, or for the general disinfection of surfaces in an area believed to be contaminated with, or at risk of contamination with, the organism. Thus the compound (III) or derivative may be used to treat an outbreak of a particular pathogen, for example a nosocomial pathogen such as S. aureus (including resistant strains such as MRSA, VISA or GISA), E. faecalis or C. difficile.

In an embodiment of the invention, the compound (III) or derivative is for use against one or more bacteria associated with skin or skin-borne infections. It may be for use against Gram-positive bacteria, for example staphylococci and/or propionibacteria, in particular against strains of Staphylococcus aureus and/or Propionibacterium acnes. In an embodiment, it is for use against one or more bacteria associated with acne, such as P. acnes and in some instances P. granulosum.

According to an embodiment of this second aspect of the invention, the compound (III) or derivative is for use in the treatment of a skin or skin structure condition. Such a condition may be a primary or secondary infection. It may for example be a superficial or uncomplicated skin infection amenable to local therapy. It may be acne or an infection associated with. It may be a primary or secondary infection due to S. aureus (including MRSA) or a group a beta haemolytic streptococcus (S. pyogenes).

The compound (III) or derivative may in particular be for use in the treatment of acne (i.e. as an anti-acne agent).

Skin and skin structure conditions which might be treated according to the invention include acne, infected atopic eczema, superficial infected traumatic lesions, wounds, burns, ulcers, folliculitis, mycoses and other primary and secondary skin and skin structure infections. In particular the compound (III) or derivative may be for use in treating acne or acne lesions (for instance, to reduce acne-related scarring).

Acne is a multifactorial disease of the pilosebaceous follicles of the face and upper trunk, characterised by a variety of inflamed and non-inflamed lesions such as papules, pustules, nodules and open and closed comedones. Its treatment can therefore encompass the treatment (which embraces prevention or reduction) of any of these symptoms, and references to use as an anti-acne agent may be construed accordingly.

In particular, the treatment of acne encompasses the treatment (including prevention) of lesions and/or scarring associated with acne. It also encompasses the treatment of a propionibacterial infection and/or the inhibition of propionibacterial activity which could cause or be otherwise associated with acne or its symptoms.

In general, the present invention will be used for the treatment of symptoms which are directly due to acne rather than for instance infections which may arise as a consequence of treating acne with other actives such as antibiotics, and/or secondary infections caused by opportunistic pathogens, which can arise in skin already affected by acne.

An additional advantage to the use of a compound (III) or derivative to treat acne and related infections can be that such compounds, as ACAT inhibitors, can be capable of inhibiting sebum secretion (see EP-0 699 439). Given the multifactorial nature of acne, a compound capable of targeting two different aspects of its pathogenesis can offer considerable advantages over one that targets only a single mechanism.

Thus, in general terms the invention can provide a compound of formula (III) or a derivative thereof, for use in the treatment of acne.

Moreover according to a further aspect of the invention, there is provided a compound of formula (III) or derivative thereof, for use in the treatment of a sebaceous gland disorder, including a condition which is caused or exacerbated by (in particular caused by) sebaceous gland secretions, for example acne. The compound (III) or derivative may be for use in the inhibition of sebum production, and/or in the inhibition of sebaceous gland secretion. In this context the compound (III) or derivative may be administered orally, transdermally or topically, preferably either orally or topically, more preferably topically.

Atopic dermatitis or eczema (atopic eczema and dermatitis syndrome AEDS), which may also be treated using the present invention, can frequently become infected by Gram-positive bacteria, most commonly by Staphylococcus aureus (David T J, 1989 Journal of the Royal Society of Medicine 82: 420-422) but also by members of the genus Streptococcus (Brook I, 2002 Journal of Medical Microbiology 51: 808-812) and possibly by members of the genus Enterococcus. The compound (III) or derivative may be for use in the treatment of infected atopic dermatitis since such compounds have been shown to be active against S. aureus (including Methicillin Resistant S. aureus (MRSA), Epidemic Methicillin Resistant S. aureus (EMRSA), Vancomycin-Intermediate Resistant S. aureus (VISA) and Glycopeptide-Intermediate Resistant S. aureus (GISA)), Streptococcus pyogenes and members of the genus Enterococcus (including E. faecalis). The compound (III) or derivative may be for use against one or more such bacteria.

Human skin is susceptible to infection by a wide range of Gram-positive bacteria. These conditions include but are not restricted to folliculitis, boils and carbuncles, impetigo most usually caused by Staphylococcus aureus and other infections including erysipelas caused by members of the genus Streptococcus, erythrasma and cellulitis caused by both staphylococci and streptococci. Other Gram-positive bacteria may also be involved in these infections including members of the genera Bacillus, Clostridium and Enterococcus. According to the invention, the compound (III) or derivative may be for use in the treatment of infected dermatoses, skin infections, superficial infected wounds and soft tissue infections since such compounds have been shown to be active against S. aureus, including MRSA, EMRSA, VISA and GISA, as well as against Streptococcus pyogenes and members of the genus Enterococcus (including E. faecalis) and members of the genus Bacillus and Clostridium.

Infected dermatoses, skin infections, superficial infected wounds and soft tissue infections may also include polymicrobial infections of the skin that are caused by both Gram-positive and Gram-negative bacteria. In such cases the compound (III) or derivative may be used in combination with an additional antimicrobial agent, for example either a topical or a systemic agent which is active against Gram-negative bacteria.

In an embodiment of the invention, the compound (III) or derivative may be for use as a treatment against staphylococci, which might otherwise cause for example MRSA-associated infections. It may be for use as a treatment against staphylococci on the skin, or in the nares, eyes or ears. The compound (III) or derivative may in particular be for use in a prophylactic treatment against staphylococci (in particular S. aureus) in the nasal carriage.

Approximately 25 to 30% of healthy individuals carry Staphylococcus aureus in the nares. The organism is also carried at other body sites and at higher prevalence in predisposed individuals such as those with atopic dermatitis. Antibiotic-resistant strains of Staphylococcus aureus (e.g. MRSA) are also widely distributed both in the hospital environment and in the community. These factors contribute to the risk of nosocomial S. aureus infections especially in patients undergoing surgery (Grundmann H, Aires-de-Sousa M, Boyce J, Tiemersma E, 2006 Lancet 368: 874-85; Herwaldt L A, 2003 Surgery 134(5 Suppl):S2-9). Compounds of formula (III) and their derivatives have been shown capable of activity against S. aureus and may be used prophylactically to eradicate and/or prevent colonisation of the nares and skin by this organism. This can be used for example in patients and hospital staff to prevent infections caused by S. aureus. The compounds are particularly well suited for this purpose as they can be active against antibiotic-resistant strains of S. aureus including MRSA, EMRSA, VISA and GISA.

According to a further embodiment of the invention, the compound (III) or derivative is for use in the treatment (which includes prevention) of body odour, for example in the axilla or feet. It may thus be for use against the bacteria implicated in this condition, in particular aerobic diphtheroids of the genus Corynebacterium.

Human body odour is formed by the action of commensal skin bacteria on the odourless secretions of sweat glands. For example the action of members of the genus Corynebacterium have been shown to release the odiferous compounds 3-hydroxy-3-methylhexonic acid and 3-hydroxy-2-methylhexonic acid from odourless precursors (Natsch A, Gfeller H, Gygax P et al, 2003 Journal of Biological Chemistry 278 (8): 5718-5727). The compound (III) or derivative may thus be for use in the prevention of body odour as such compounds have been shown to be active against species of the genus Corynebacterium, including corynebacteria of human origin such as C. mucifaciens. Such compounds have also been shown to be active against other members of the bacterial human skin microflora such as cutaneous propionibacteria, and coagulase negative staphylococci that may also contribute to human body odour. According to the invention, the compound (III) or derivative may therefore be used against one or more such bacteria.

In an embodiment, the compound (III) or derivative is for use in the treatment of a microbial condition within the oral cavity. It may therefore be for use against one or more bacteria associated with conditions affecting the oral cavity, for example dental caries, periodontal diseases or halitosis.

Thus for example, the compound (III) or derivative may be for use against strains of P. gingivalis. Furthermore, such compounds have been found to be active against Gram-positive and Gram-negative bacteria known to be associated with dental plaque and the formation of biofilms which have been implicated in the etiology of periodontal diseases, including gingivitis. The compound (III) or derivative may therefore be of use against, among others, the following Gram-positive bacteria: Streptococcus mutans, Streptococcus sobrinus and Actinomyces naeslundii, and surprisingly the following Gram-negative bacteria: Porphyromonas gingivalis and Prevotella nigrescens, all of which can contribute to the microflora found in dental biofilms involved in periodontal disease.

The compound (III) or derivative may be for use in the treatment of a periodontal disease, and/or as an anti-plaque agent.

Periodontal diseases which may be treated using the present invention (the term “treat” embracing both therapeutic and prophylactic measures, as defined above) include for example dental plaque-induced gingival diseases; chronic (previously adult) periodontitis; aggressive periodontitis (formerly early-onset, prepubertal, juvenile or rapidly progressive periodontitis); necrotising periodontal diseases; abcesses of the periodontium; and post-operative bacterial infections (in particular those which are caused, transmitted and/or exacerbated by P. gingivalis). The invention may also be used to treat bacterial infections (in particular those which are caused, transmitted and/or exacerbated by P. gingivalis) of wounds or other lesions within the oral cavity, including those arising due to other medical conditions such as oral candidiasis.

Associated symptoms which may be treated using the invention include mouth ulcers, dental pain, discomfort, inflammation, bleeding, pus secretion, halitosis, tooth mobility, tooth loss, swelling or inflammation caused by any of the foregoing.

Since plaque formation on tooth surfaces can also cause, exacerbate or accompany periodontal diseases, in accordance with the present invention the compound (III) or derivative may be used to reduce or prevent plaque formation, and/or to alter (suitably beneficially) the bacterial composition of plaque.

It has further been found that periodontal inflammatory diseases may be linked to (i.e. may in cases cause, increase susceptibility to and/or exacerbate) other more serious, often more systemic conditions. For example, periodontal diseases, and/or the by-products generated by associated pathogens such as P. gingivalis, have been linked with coronary artery disease and other cardiovascular diseases such as myocardial infarction, atherosclerosis and angina, as well as with conditions associated with arterial inflammation or blood clot formation and with an increased risk of pre-term low-weight births (Gotsman et al, J. Periodontol. May 2007, 78(5): 849-858; Noack et al, J. Periodontol. 2001, 72: 1221-1227; Seymour et al, Clinical Microbiology and Infection, 13 (Suppl 4): 3-10). The present invention may accordingly be used, indirectly, to treat any such condition as well as to treat infections within the oral cavity and associated periodontal diseases.

It has also been recognised that periodontal diseases can pose a threat to the health of those suffering from chronic diseases such as diabetes, respiratory diseases, osteoporosis and AIDS (Kuo et al, Seymour et al, supra). Thus the present invention may be used to reduce health risks to such patients from actual or potential periodontal infections.

Instead or in addition, the compound (III) or derivative may be for use in the treatment of dental caries. It may be for use against mutans streptococci, for example strains of S. mutans. It may be for use against a condition within the oral cavity which is caused, exacerbated or transmitted by (in particular either caused or transmitted by) such bacteria.

It is well known that mutans streptococci (MS) such as Streptococcus mutans and related bacteria (e.g. Streptococcus sobrinus) are implicated as causative agents of dental caries (Loesch W J, 1986 Microbiological Reviews 50(4): 353-380; Islam B, Khan S N, Khan, 2007 Medical Science Monitor 13(11):196-203). This is particularly the case in children and other individuals that have a high sugar diet.

MS, especially S. mutans, are acid-tolerant and are highly cariogenic. They induce the formation of dental caries by the production of short chain carboxylic acids that demineralise and damage both tooth enamel and dentine. This effect is enhanced by close association between MS and the tooth surface in the form of a biofilm. The attachment of MS to the tooth surface is facilitated by their ability to produce extracellular polysaccharides such as glucans as a result of sugar metabolism. The attachment of Streptococcus mutans is further enhanced by the production of a specific adhesion protein (often referred to as PAc, antigen I/II, PI, and Spa PI), that is involved in both its primary attachment to the tooth and in providing further bacterial attachment sites. These factors contribute to the formation of a multi-species bacterial biofilm on the tooth surface. The biofilm is formed firstly by deposition of an acquired enamel pellicle comprising salivary and bacterial components, then adherence and co-adherence of multiple bacterial species of the oral cavity (including Actinomyces naeslundii) and finally the proliferation of these organisms. The biofilm provides a niche environment in which the cariogenic MS can thrive. It is notable that co-colonisation of the tooth surface by S. mutans and S. sobrinus leads to increased cariogenicity (Okada M, Soda Y, Hayashi F, 2005 Journal of Medical Microbiology 54: 661-665).

The present invention may be used to inhibit the growth of MS in the oral cavity and thereby reduce the cariogenic effects of these organisms. Furthermore the invention may be used to inhibit other bacterial components of the biofilm (for example Actinomyces naeslundii) adhering to the tooth surface, thus reducing biofilm integrity and its ability to provide a niche environment for cariogenic MS.

It has further been found that MS may be linked to (i.e. may in cases cause, increase susceptibility to and/or exacerbate) other more serious, often more systemic conditions. For example, MS have been linked with bacteraemia and its sequlae including but not limited to acute and subacute endocarditis (Verghagen D W M, Vedder A C, Speelman P, van der Meer, 2006 Journal of Antimicrobial Chemotherapy 57: 819-824). The present invention may accordingly be used, indirectly, to treat any such condition as well as to treat infections within the oral cavity and associated dental caries.

In an embodiment, the compound (III) or derivative may be for use in the treatment of an ocular infection. It may for example be used in the treatment of conjunctivitis due to Corynebacterium spp or in particular S. aureus, or in the treatment (in particular the prevention) of endophthalmitis due to Propionibacterium spp.

In an embodiment, the compound (III) or derivative may be for use in the treatment of an infection within the ear.

Infections of the eye and ear are commonly caused by Gram-positive bacteria (Kowalski R P, Dhaliwal D K, 2005 Expert Rev Anti Infect Ther. 3(1):131-9; de Miguel Martinez I, Ramos Macias A, Masgoret Palau E, 2007 Acta Otorrinolaringol Esp. 58(9):408-12). They are often caused by staphylococci, streptococci and occasionally by cutaneous propionibacteria. The compound (III) or derivative may be for use in the treatment of infections of the eye or ear since such compounds have been shown to be active against the following Gram-positive bacteria that are commonly identified as causative agents: Staphylococcus aureus (including MRSA, EMRSA, VISA and GISA), coagulase-negative staphylococci including S. auricularis, streptococci such as Streptococcus pyogenes and propionibacteria such as Propionibacterium acnes and Propionibacterium granulosum.

Infections of the eye or ear may also be caused by Gram-negative bacteria. In order to extend the activity, against such infections, of a formulation containing a compound (III) or derivative thereof, it may contain, or be used in combination with, another antimicrobial agent, in particular a topical or systemic agent which is active against Gram-negative bacteria.

In an embodiment, the compound (III) or derivative may be for use in the treatment of a bacterial condition affecting the vagina, for example vaginitis or vaginosis. It may be for use against a bacterium implicated in such a condition, for example Gardnerella vaginalis which is implicated in vaginitis and vaginosis, or other anaerobic bacteria such as Bacteroides spp. It may be for use in the treatment of a sexually transmitted disease, for example a disease in which Gardnerella vaginalis and/or Neisseria gonorrhoae are implicated.

The compound (III) or derivative may be for use in the treatment of a urinary tract infection (UTI). Such infections are caused by either Gram-positive bacteria, Gram-negative bacteria or mixed populations containing both types of bacteria. Among the Gram-positive organisms frequently implicated in UTIs are Staphylococcus aureus, coagulase negative staphylococci and enterococci. The compound (III) or derivative may be for use in the treatment of UTIs since such compounds have been shown to be active against the following Gram-positive bacteria that are commonly identified as causative agents of UTIs: S. aureus (including MRSA, EMRSA, VISA and GISA), coagulase-negative staphylococci (including S. saprophyticus, S. cohnii, S. capitis, S. epidermidis, S. haemolyticus, S. hominis and S. xylosus) and also members of the genus Enterococcus (including E. faecalis).

In an embodiment, the compound (III) or derivative may be for use in the treatment of an infection associated with an indwelling surgical device or implant, for example a catheter. Bacterial infections can frequently arise through the use of such devices (Wenzel R P, 2007 CID 45 (Suppl 1): S85-S88). This is particularly the case when Staphylococcus aureus, coagulase-negative staphylococci, streptococci, cutaneous propionibacteria (e.g. in the case of artificial hip joints) and other bacteria adhere to the device and form a focus of infection and/or biofilm. Bacteria can detach from the initial infectious site and may be linked to (i.e. may in cases cause, increase susceptibility to and/or exacerbate) other, systemic conditions such as bacteraemia and its sequlae, including for example acute and subacute endocarditis. Thus the compound (III) or derivative may be for use in the treatment of such infections and/or associated conditions.

Compounds of formula (III) and their derivatives may thus be used in the treatment of infections associated with indwelling surgical devices, since such compounds have been shown to be active against S. aureus (including MRSA, EMRSA, VISA and GISA), coagulase-negative staphylococci (including but not restricted to S. auricularis, S. capitis, S. cohnii, S. epidermidis, S. haemolyticus, S. hominis, S. saprophyticus, S. simulans, S. warneri and S. xylosus), members of the genus Enterococcus (including E. faecalis), members of the genus Streptococcus (including. S. pyogenes), propionibacteria such as Propionibacterium acnes and Propionibacterium granulosum and members of the genus Corynebacterium (including C. mucifaciens).

Infections associated with catheters and other indwelling surgical devices may also be polymicrobial infections caused by both Gram-positive and Gram-negative bacteria. In such cases the compound (III) or derivative may be used in combination with another antimicrobial agent, in particular a topical or systemic agent which is active against Gram-negative bacteria.

In an embodiment, the compound (III) or derivative may be for use in the treatment of an infection of a wound (in particular a deep-seated wound), burn or ulcer. Deep-seated wounds, burns and ulcers are often infected by either Gram-positive bacteria, Gram-negative bacteria or mixed populations containing both types of bacteria (Hedrick T L, Smith P W, Gazoni L M et al, 2007, Current Problems in Surgery 44(10):635-75; Meara S O, Cullum, N, Majid M et al, 2000 Health Technology Assessment 4(21); Church D, Elsayed S, Reid O, 2006 Clinical Microbiology Reviews 19(2): 403-434; Anderson C A, Roukis T S, 2007 Surgical Clinics of North America 87:1149-1177). Compounds of formula (III) and their derivatives have been found capable of activity against a wide range of Gram-positive bacteria involved in such infections, and against certain anaerobic Gram-negative bacteria which can also be involved in these infections.

The compound (III) or derivative may be for use in the treatment of infected deep-seated wounds, burns and ulcers since such compounds have been shown to be active against the following Gram-positive bacteria that are commonly identified as the infective agents: Staphylococcus aureus (including MRSA, EMRSA, VISA and GISA), coagulase-negative staphylococci (including but not restricted to S. auricularis, S. capitis, S. cohnii, S. epidermidis, S. haemolyticus, S. hominis, S. saprophyticus, S. simulans, S. warneri and S. xylosus), members of the genus Streptococcus (e.g. S. pyogenes), members of the genus Enterococcus (including E. faecalis), members of the genus Bacillus (e.g. B. cereus), members of the genus Clostridium, propionibacteria such as Propionibacterium acnes and Propionibacterium granulosum, and members of the genus Corynebacterium (e.g. C. mucifaciens). Surprisingly a compound (III) or derivative can also possess activity against the Gram-negative anaerobes Porphyromonas gingivalis and Prevotella nigrescens, which also may be involved in infections of deep-seated wounds, burns and ulcers.

In an embodiment, the compound (III) or derivative is for use in the treatment of an infection within the throat. Throat infections can be caused by streptococci such as Streptococcus pyogenes or for instance by staphylococci such as Staphylococcus aureus (including MRSA, EMRSA, VISA and GISA). They may therefore be treated using a compound (III) or derivative since such compounds have been shown capable of activity against these causative organisms. A compound (III) or derivative may for example be applied to a catheter, or incorporated into a mouthwash or other oral health care product, for the purpose of preventing, or reducing the risk of, a throat infection.

In an embodiment, the compound (III) or derivative may be for use in the treatment, in particular the prophylaxis, of an opportunistic infection. Immuno-compromised individuals who are otherwise susceptible to infection, for example due to HIV infection or other underlying diseases, malnutrition or the administration of immunosuppressive drugs, can be predisposed to opportunistic bacterial infections. Such infections can be caused by a wide range of Gram-positive and Gram-negative bacteria. Compounds (III) and their derivatives have been found capable of activity against a wide range of Gram-positive bacteria involved in such infections and against certain anaerobic Gram-negative bacteria also involved in these infections. Thus a compound (III) or derivative thereof may be for use in the treatment of opportunistic infections since such compounds have been shown to be active against the Gram-positive bacteria which are commonly identified as the infective agents, for example: Staphylococcus aureus (including MRSA, EMRSA, VISA and GISA), coagulase-negative staphylococci (including S. auricularis, S. capitis, S. cohnii, S. epidermidis, S. haemolyticus, S. hominis, S. saprophyticus, S. simulans, S. warneri and S. xylosus), members of the genus Streptococcus (including S. pyogenes, S. mutans, S. sobrinus and S. pneumoniae), members of the genus Enterococcus (including E. faecalis), members of the genus Bacillus (e.g. B. cereus), members of the genus Clostridium (e.g. C. difficile or C. sporogenes), cutaneous propionibacteria such as Propionibacterium acnes and Propionibacterium granulosum, members of the genus Corynebacterium (including C. mucifaciens) and members of the family Actinomycetes (e.g Actinomyces naeslundii). For this purpose the compound (III) or derivative may for instance be applied to a dressing, surgical instrument, implant, catheter or the like to reduce the risk of bacterial infection during or after use of the item.

It has surprisingly also been found that compounds of formula (III) and their derivatives can possess activity against the Gram-negative anaerobes Porphyromonas gingivalis and Prevotella nigrescens, which also may be involved in opportunistic infections.

In an embodiment, the compound (III) or derivative may be for use in preventing the transmission of a food-borne bacterial pathogen, for example an infection caused by S. aureus, B. cereus, E. faecalis or Listeria monocytogenes. Moreover, as discussed below, in accordance with the invention the compound (III) or derivative may be used to control bacterial growth in order to inhibit, prevent or reduce food spoilage. Such compounds have been shown to be active, or by inference from experimental data are believed to be active, against the following Gram-positive bacteria that are commonly identified as food spoilage agents and food borne pathogens: Staphylococcus aureus (including MRSA, EMRSA, VISA and GISA), members of the genus Streptococcus, members of the genus Enterococcus (including E. faecalis and Enterococcus faecium), members of the genus Bacillus (e.g. B. cereus), members of the genus Clostridium (e.g. C. sporogenes), members of the genus Lactobacillus and Listeria monocytogenes (sole species).

In an embodiment, the compound (III) or derivative may be for use in the disinfection of skin or other tissue surfaces. Moreover, as described below, it may be used for the disinfection of non-living areas and surfaces. It may in particular be used to counter bacteria of the type referred to above. For example, it may be used to disinfect against the following Gram-positive bacteria: Staphylococcus aureus, members of the genus Enterococcus (including E. faecalis and E. faecium), members of the genus Bacillus (e.g. B. cereus), members of the genus Clostridium (e.g. C. sporogenes) and Listeria monocytogenes.

Surprisingly, compounds of formula (III) and their derivatives have also been found capable of activity against the Gram-negative anaerobes Porphyromonas gingivalis and Prevotella nigrescens, and may therefore be used to disinfect against these organisms.

In an embodiment, the compound (III) or derivative may be for use in the treatment of a bacterial condition affecting an epithelial or mucosal surface such as in the nares, scalp, vagina, eyes, ears or oral cavity.

In an embodiment, the compound (III) or derivative is for use in the treatment of a condition which is caused, transmitted and/or exacerbated by (in particular either caused or transmitted by) a Gram-positive bacterium (whether aerobic or anaerobic), and/or by a bacterium selected from the group consisting of Gram-positive bacteria, P. gingivalis, Prevotella nigrescens and Gardnerella vaginalis.

In cases the compound (III) or derivative may be for use in the treatment of a condition which is caused, transmitted and/or exacerbated by (in particular either caused or transmitted by) an aerobic Gram-negative bacterium.

In an embodiment, the compound (III) or derivative is for use in the treatment of a condition selected from skin and skin structure conditions, in particular acne or superficial skin infections due to staphylococci and/or streptococci; body odour; staphylococcal infections (in particular by S. aureus); ocular infections such as conjunctivitis; infections caused by nosocomial pathogens such as S. aureus, E. faecalis or C. difficile; and periodontal diseases.

In an embodiment, the compound (III) or derivative is for use against one or more bacteria selected from staphylococci (in particular S. aureus and in cases also coagulase-negative staphylococci such as S. auricularis, S. capitis, S. cohnii, S. epidermidis, S. haemolyticus, S. hominis, S. saprophyticus, S. simulans, S. warneri and S. xylosus), members of the genus Streptococcus, members of the genus Enterococcus (in particular E. faecalis), members of the genus Bacillus (e.g. B. cereus), members of the genus Clostridium (in particular C. difficile), cutaneous propionibacteria (in particular P. acnes) and members of the genus Corynebacterium.

In an embodiment, the compound (III) or derivative is for use against one or more bacteria selected from staphylococci, members of the genus Streptococcus, members of the genus Enterococcus, members of the genus Clostridium (in particular C. difficile), cutaneous propionibacteria (in particular P. acnes) and members of the genus Corynebacterium.

In an embodiment, the compound (III) or derivative is for use against one or more bacteria selected from staphylococci, members of the genus Enterococcus (in particular E. faecalis), members of the genus Clostridium (in particular C. difficile), cutaneous propionibacteria (in particular P. acnes) and members of the genus Corynebacterium.

In an embodiment, the compound (III) or derivative is for use in the treatment of a condition selected from acne, body odour and S. aureus infections (in the latter case, it may in particular be used for the prophylaxis of S. aureus in the nasal carriage).

In certain cases, the compound (III) or derivative is not for use in the treatment of a polymicrobial or mixed infection, including in cases an infection which involves a Gram-negative bacterium. In certain cases, it is not for use against a Gram-negative bacterium. In certain cases, it is not for use in the topical treatment of burns and/or of deep wounds.

In accordance with the invention, the compound (III) or derivative is preferably applied topically, to either a living or a non-living surface as appropriate. It is preferably used in the form of a formulation which is suitable for topical application to, and/or contact with, the skin, in particular human skin. It is therefore preferably contained in a pharmaceutically acceptable vehicle which can safely be applied to, and/or contacted with, the skin. Ideally the formulation is suitable for topical application to areas such as the nares, eyes, scalp and/or vagina, and/or to tissue within the ears and/or the oral cavity. It may be suitable for topical application to tissue surfaces within the nares and/or ears, in particular the nares. It may be suitable for topical application to one or more tissue surfaces within the mouth, in particular the gums, gingivae, periodontium and/or teeth. It may in particular be suitable for topical application to such surfaces in or on the human body.

A formulation which is “suitable for” topical application may also be adapted for topical application.

A formulation which is applied topically within the oral cavity will not, in the course of its ordinary usage, be intentionally swallowed for the purpose of systemically administering a substance contained within it, but rather, will be retained in the oral cavity, in contact with the relevant oral tissue and/or dental surface, for sufficient time as to exert a pharmaceutical effect, for example an antibacterial and/or anti-plaque effect.

Suitable vehicles for topical formulations will be well known to those skilled in the art of preparing topical skin care or pharmaceutical preparations. The vehicle will typically be a fluid, which term includes a cream, paste, gel, lotion, foam, ointment, varnish or other viscous or semi-viscous fluid, as well as less viscous fluids such as might be used in sprays (for example for nasal or oral use), drops (for example for use in the eyes or ears), aerosols or mouthwashes. The compound (III) or derivative may be present in the form of a solution or suspension, the term “suspension” including emulsions, micellar systems and other multi-phase dispersions.

The compound (III) or derivative may be carried in or on a delivery vehicle which is suitable for targeting or controlling its release at the intended site of administration. Such vehicles include liposomes and other encapsulating entities, for example niosomes, aspasomes, microsponges, microemulsions, hydrogels and solid lipid nanoparticles.

A third aspect of the present invention provides the use of a compound of formula (III), or a pharmaceutically acceptable derivative thereof, in the manufacture of a medicament (typically a formulation) for the treatment of a condition which is caused by, transmitted by and/or exacerbated by (in particular either caused or transmitted by) bacterial activity. Again the condition may be selected from those listed above in connection with the first and second aspects of the invention. It may be a skin or skin structure condition, in particular acne. It may be a staphylococcal infection. It may be body odour. It may be a condition affecting the oral cavity, in particular a periodontal disease. The compound (III) or derivative will typically be used as an antibacterial agent in the manufacture of the medicament.

The invention also provides the use of a compound of formula (III), or a pharmaceutically acceptable derivative thereof, in the manufacture of a medicament (typically a formulation) for the treatment of a sebaceous gland disorder, as described above in connection with the first aspect of the invention. The medicament may be for use in the inhibition of sebum production, and/or in the inhibition of sebaceous gland secretion.

In the compound of formula (III), R¹ and R² may be either methyl or ethyl, most preferably methyl. R¹ and R² are suitably the same.

In an embodiment of the invention, the compound of formula (III) is selected from Antioxidant 2246 (AO 2246, of formula (I) above); Antioxidant 425 (AO 425, of formula (II) above); and mixtures thereof.

In another embodiment, the compound of formula (III) is AO 2246.

The compound (III) or derivative, in particular AO 2246, is ideally used in the form of the isolated compound (whether naturally or synthetically derived, preferably the latter) rather than as part of a plant extract containing a number of different materials. It is thus suitably used in a substantially pure form, for instance containing 1% w/w or less, preferably 0.5 or 0.2 or 0.1% w/w or less, in cases 0.05% w/w or less, of impurities.

A “derivative” of a compound of formula (III), for example of AO 2246, may be a pharmaceutically acceptable (which term includes acceptable for veterinary use) derivative. It may be for example a salt, complex or solvate or a so-called “prodrug” form or protected form which reverts to an active form of the relevant compound at an appropriate time on or after administration. Preferably, however, the compound (III) is present in the form of a single, underivatised molecule of the formula (III) defined above.

A formulation prepared or used according to the invention may contain a mixture of two or more different compounds of formula (III) or derivatives thereof.

In accordance with the invention, the compound (III) or derivative is used as an antibacterially active agent. Instead or in addition, it may be used as an anti-acne agent (i.e. as an agent which is active against acne (which includes against a symptom and/or a cause of acne) and/or against one or more micro-organisms associated with acne). It may be used as an anti-plaque agent, as described above. It is not used purely or even primarily as an antioxidant, or as a stabiliser for another substance such as an active ingredient, or as a preservative in a product containing another substance such as an active ingredient. Nor is it used purely or even primarily as a free radical polymerisation inhibitor. Nor is it used purely or even primarily for modulating cellular immune reactions, for reducing serum lipid (including cholesterol) levels, for suppressing macrophage foaming, as an anti-hyperlipidemia agent, as an anti-arteriosclerosis agent or as an anti-tumour or anti-cancer agent. In an embodiment of the invention, the compound (III) or derivative is not used purely or even primarily as an antiviral agent. In an embodiment, it is not used purely or even primarily as an antifungal agent.

Antibacterial activity may be growth inhibitory activity or more preferably bactericidal (i.e. lethal to the relevant organism). It may comprise activity against sessile and/or planktonic bacteria.

In the context of this invention, activity against a particular species of micro-organism may be taken to mean activity against at least one, preferably two or more, strains of that species.

Antibacterial activity may be or include the ability to disrupt and/or suppress biofilm formation by the relevant organism; thus, in accordance with the invention, the compound (III) or derivative may be used to treat a condition which is caused, transmitted and/or exacerbated by bacterial biofilm formation.

In the present context, the disruption of biofilm formation embraces any negative effect on the ability of a micro-organism to form, maintain or exist in a biofilm, and/or on a biofilm already formed by the organism. Thus, it may involve reducing the amount of a previously formed biofilm, and/or impairing such a biofilm. It may involve killing or inhibiting sessile bacteria within a biofilm.

The biofilm may for example be in the form of dental plaque. The disruption may involve altering the bacterial composition of dental plaque to a more healthy state, reducing the amount of plaque by for example inhibiting co-aggregation, and/or interfering with the production of signal molecules that mediate the organisation and integrity of plaque.

Suppression of biofilm formation embraces any degree of impairment (including complete prevention) of the ability of a micro-organism to form, or more typically to co-aggregate with, a biofilm. It thus embraces total or partial impairment, including reducing the amount and/or strength of biofilm which the organism is able to form and/or the speed with which it is able to do so. It may involve preventing or reducing the growth or the rate of growth of an existing biofilm formed by the organism.

An antibacterial formulation prepared or used according to the present invention is preferably active at least as a bactericide, more preferably against Gram-positive bacteria, for example against one or more bacteria selected from Propionibacterium spp, staphylococci and bacteria implicated in body odour. It may in particular be active against one or more of the micro-organisms referred to above in connection with the first to the third aspects of the invention.

In a particularly preferred embodiment of the invention, the formulation is active against one or more bacteria associated with acne, such as P. acnes and in some instances P. granulosum. In another preferred embodiment, the formulation is active against one or more bacteria associated with conditions affecting the oral cavity, for example S. mutans or in particular P. gingivalis. In another preferred embodiment, the formulation is active against one or more staphylococci, in particular S. aureus. In another preferred embodiment, the formulation is active against one or more bacteria associated with body odour, in particular in the axilla or feet.

Such a formulation is preferably active against micro-organisms, in particular staphylococci and/or propionibacteria, which are wholly or partially resistant to one or more antibiotics, for instance those which are in common clinical use. The formulation is ideally active against MRSA bacterial strains, for example, and/or against other resistant staphylococci such as VISA or GISA. It may be active against one or more multiresistant bacteria such as E. faecalis. It may be active against one or more oral streptococci.

Instead or in addition, the formulation is ideally active against macrolide-lincosamide-streptogramin (MLS) resistant and/or macrolide-lincosamide-streptogramin-ketolide (MLSK) resistant bacteria. In particular it may be active against erythromycin-resistant, clindamyc in-resistant and/or tetracycline-resistant P. acnes strains of bacteria, the term tetracycline here referring to the class of antibiotics including for example minocycline and doxycycline as well as the specific antibiotic known as tetracycline.

In order to extend the activity, for instance against other Gram-negative bacteria, of a formulation containing a compound (III) or derivative thereof, it may contain, or be used in combination with, an additional antimicrobial agent, in particular an agent which is active against Gram-negative bacteria. Its activity spectrum may be further extended by the inclusion of, or its use in combination with, one or more antimicrobial agents which are active against pathogenic microfungi. An additional antimicrobial agent may be for either topical or systemic administration.

Antibacterial activity may be measured in conventional manner, for instance using the tests described in the examples below. Generally tests for activity involve treating a culture of the relevant micro-organism with the candidate antibacterial compound, incubating the treated culture under conditions which would ordinarily support growth of the micro-organism, and assessing the level of growth, if any, which can occur in the presence of the candidate compound.

Preferably the compound (III) or derivative has a minimum inhibitory concentration (MIC), of 125 μg/ml or less, more preferably 62.5 μg/ml or less, yet more preferably 31.25 or 15.6 μg/ml or less, most preferably 10 or 5 or 4 μg/ml or less. Its corresponding minimum biocidal concentration (MBC) is preferably 250 μg/ml or less, more preferably 125 μg/ml or less, yet more preferably 62.5 or 31.25 μg/ml or less, most preferably 15.6 μg/ml or less. Suitably the ratio of its MIC to its MBC is from 0.01 to 1 or from 0.125 to 1, ideally from 0.5 to 1.

Preferably the compound (III) or derivative has a minimum inhibitory concentration (MIC), at least against propionibacteria and/or staphylococci, of 50 μg/ml or less, more preferably 10 μg/ml or less, most preferably 5 or 2 μg/ml or less. Its corresponding minimum biocidal concentration (MBC) is preferably 100 μg/ml or less, more preferably 70 or 50 μg/ml or less, most preferably 10 or 5 μg/ml or less. Suitably the ratio of its MIC to its MBC, in this context, is from 0.01 to 1 or from 0.125 to 1, ideally from 0.5 to 1. More preferably the compound (III) or derivative also exhibits such characteristics in the presence of at least one of, preferably two or more of, lipid, salt (sodium chloride) and blood—these are species which can be present at the surface of the skin and hence performance in this context can be indicative of suitability for use in topical skin treatment formulations. Activity in the presence of lipid and sodium chloride can be especially important in the context of acne treatment.

The concentration of the compound (III) or derivative in a formulation prepared or used according to the invention might suitably be 0.1 or 0.2% w/v or greater, preferably 0.5% w/v or greater. Its concentration might be up to 5% w/v, preferably up to 2% w/v, such as from 0.5 to 2% w/v or from 1 to 2% w/v. In cases, for instance when the compound (III) or derivative is for use in the disinfection of a tissue surface such as the skin, its concentration in the formulation may be for example from 1 to 5% w/v or from 2 to 5% w/v.

As described above, a formulation prepared or used according to the invention is preferably suitable for, and more preferably adapted for, topical administration to human or animal, in particular human, skin. It may be suitable for, and more preferably adapted for, topical administration to the teeth, gums, gingivae, periodontium, tongue, skin or other tissue surfaces within the human or animal, in particular human, mouth. It may be suitable for, and/or adapted for, topical administration to other epithelia such as the nares, scalp, ears, eyes and/or vagina, in particular the nares and ears.

Such a formulation may take the form of a lotion, cream, ointment, varnish, foam, paste or gel, or any other physical form known for topical administration. It may comprise a formulation which is, or may be, applied to a carrier such as a sponge, swab, brush, tissue, cloth, wipe, skin patch, dressing (which includes a bandage, plaster, skin adhesive or other material designed for application to a tissue surface, in particular to a wound) or dental fibre to facilitate its topical administration. It may comprise a formulation which is, or may be, carried on or in an implant (including for example a chip for insertion into a periodontal pocket, or a dental filling, bridge or cap), an impregnated dental fibre or a chewing gum, tablet or chewable capsule. It may be intended for pharmaceutical (which includes veterinary but is preferably human) use, and/or for cosmetic or other non-medical care purposes (for example, for general hygiene or skin cleansing or for improving the appearance of the teeth or gums).

For use in the treatment of a condition affecting the oral cavity, the formulation may in particular take the form of a toothpaste, mouthwash, dentifrice, lozenge or buccal patch, or it may be carried in or on a dental fibre or tape.

The vehicle in which the compound (III) or derivative is contained may be any vehicle or mixture of vehicles which is suitable for topical application; the type chosen will depend on the intended mode and site of application. Many such vehicles are known to those skilled in the art and are readily available commercially. In the context of formulations for topical application to the skin, examples may for instance be found in Williams' “Transdermal and Topical Drug Delivery”, Pharmaceutical Press, 2003, and other similar reference books. See also Date, A. A. et al, Skin Pharmacol. Physiol., 2006, 19(1): 2-16 for a review of topical drug delivery strategies, and also “Skin Delivery Systems”, 2006, John J. Wille, Ed., Blackwell Publishing; “Textbook of Cosmetic Dermatology”, 2004, 3^{rd} edition, Robert Baran, Howard I Maibach, Taylor & Francis; and “Skin Care Beyond the Basics”, 2001, Mark Lees, Milady.

In the context of formulations for topical application within the oral cavity, examples of suitable vehicles may be found in “Oral Hygiene Products and Practice”, 1988, Morton Prader, Ed., Marcel Dekker, Inc., New York, N.Y., USA.

In the context of formulations for ocular use, examples of suitable vehicles may be found in “Ocular Therapeutics and Drug Delivery: A Multidisciplinary Approach”, 1995, Indra K. Reddy, Ed., CRC Press; and “Ophthalmic Drug Delivery Systems”, 2003, Ashim K. Mitra, Ed., Informa Healthcare.

Also as described above, the vehicle may be such as to target a desired site and/or time of delivery of the formulation. It may for instance target the formulation to the skin or hair follicles or to the anterior nares (the latter being particularly suitable when the formulation is used as a preventative treatment against staphylococci), most preferably to the skin or hair follicles. It may target the formulation to the gums or teeth or other areas within the oral cavity. It may delay or otherwise control release of the formulation over a particular time period. The compound (III) or derivative may be microencapsulated, for instance in liposomes—particularly suitable liposomes, for topical application to the skin, are those made from stratum corneum lipids, e.g. ceramides, fatty acids or cholesterol.

In some cases a polar vehicle may be preferred. Where the formulation is intended for use on the skin, the vehicle may be primarily non-aqueous, although in the case of an anti-acne treatment an aqueous vehicle may be used. The vehicle may be surface-active, in particular when it is intended for use in treating surfaces, for instance to cleanse instruments or working areas in particular against staphylococci. It is suitably volatile. In cases the vehicle may be alcohol-based or silicon-based.

By way of example, a lotion or gel formulation intended for application to the skin may contain a mixture of water, an alcohol such as ethanol or phenoxyethanol and a glycol such as propylene glycol.

The formulation may contain standard excipients and other additives known for use in pharmaceutical or veterinary formulations. For example, where the formulation is intended for topical application to the skin, in particular to treat skin and skin structure conditions and/or to treat conditions such as acne or atopic dermatitis, examples of suitable excipients and additives include emollients, perfumes, antioxidants, preservatives, stabilisers, gelling agents and surfactants; others may be found in Williams' “Transdermal and Topical Drug Delivery”, supra. For the treatment of acne, however, it may be preferred for the formulation not to contain an emollient.

Such a formulation may further contain additional active agents such as antimicrobial (in particular antibacterial) agents. For example, it may contain one or more agents selected from anti-acne agents, keratolytics, comedolytics, anti-inflammatories, anti-proliferatives, antibiotics, anti-androgens, sebostatic agents, anti-pruritics, immunomodulators, agents which promote wound healing and mixtures thereof; it may instead or in addition contain one or more agents selected from sunscreens, moisturisers, emollients and mixtures thereof.

An additional antimicrobial agent may for example be selected from the group consisting of biocides, disinfectants, antiseptics, antibiotics, bacteriophages, enzymes, anti-adhesins, immunoglobulins, other antimicrobially active antioxidants and mixtures thereof; it is preferably active as a bactericide, in particular against propionibacteria and/or staphylococci.

It may however be preferred for the compound (III) or derivative to be the only active agent in the formulation, or at least to be the only antimicrobially or antibacterially active agent and/or the only anti-acne active agent.

In the case where the formulation is intended for topical application within the oral cavity, suitable excipients and additives include those known for use in oral health care formulations. Examples include flavourings, antioxidants, preservatives, stabilisers, gelling agents and surfactants; others may be found in “Oral Hygiene Products and Practice”, 1988, supra. Surfactants may be particularly preferred, as they can help to disrupt, and/or prevent formation of, microbial biofilms.

Such a formulation should contain an orally acceptable and systemically non-toxic vehicle. For example, where it takes the form of a toothpaste, a typical vehicle might include water and a humectant to provide a liquid base, together with one or more of a thickener, a surfactant and a polishing agent. Suitable humectants include glycerol, sorbitol and polyethylene glycol, and in particular mixtures thereof. A polyethylene glycol humectant may for example have a molecular weight range of from 200 to 1000 or from 400 to 800.

Suitable thickeners for use in toothpaste formulations include natural and synthetic gums and colloids such as carrageenan, xanthan gum and sodium carboxymethyl cellulose, as well as gum tragacanth; starch; polyvinyl pyrrolidone; cellulosic thickeners such as hydroxyethyl propyl cellulose, hydroxybutyl methyl cellulose, hydroxypropyl methyl cellulose, hydroxyethyl cellulose and water soluble salts of cellulose ethers such as sodium carboxymethyl cellulose or sodium carboxymethyl hydroxyethyl cellulose; and carboxyvinyl polymers. Suitable inorganic thickeners include colloidal silica, colloidal magnesium aluminium silicate, finely divided silica and synthetic hectorite. Mixtures of thickeners may also be used.

Suitable surfactants for use in toothpaste formulations prepared according to the invention include water soluble detergents. In general they may be anionic, nonionic, cationic, zwitterionic, amphoteric or ampholytic, but are preferably anionic. Examples of suitable anionic surfactants include higher alkyl sulphates such as sodium lauryl sulphate, and higher fatty acid esters of 1,2 dihydroxy propane sulphonate. Examples of suitable water soluble nonionic surfactants include the polymeric condensation products of hydrophilic alkylene oxide group-containing compounds (typically ethylene oxide) with organic hydrophobic compounds (for example those having aliphatic chains of about 12 to 20 carbon atoms). Such products include the “ethoxamers” and include for example the condensation products of poly(ethylene oxide) with fatty acids, fatty alcohols, fatty amides and other fatty moieties, as well as with propylene oxide and polypropylene oxides (the latter being available, for example, under the trade name Pluronic®).

A toothpaste will suitably contain an abrasive or polishing agent. Suitable such agents include siliceous materials (including gels and precipitates, such as precipitated amorphous hydrated silicas, aluminium silicate, zirconlure silicate, silica gel and colloidal silica); carbonates and bicarbonates such as calcium carbonate and sodium bicarbonate; phosphates such as sodium metaphosphate, potassium metaphosphate, tricalcium phosphate, dicalcium orthophosphate dehydrate, calcium phosphate dihydrate, anhydrous dicalcium phosphate, calcium pyrophosphate, calcium to polymetaphosphate, magnesium orthophosphate, trimagnesium phosphate and insoluble sodium polymetaphosphate; alumina trihydrate; calcined alumina; bentonite; complex amorphous alkali metal aluminosilicates; and resinous abrasive materials such as particulate condensation products of urea and formaldehyde. Others are disclosed in U.S. Pat. No. 3,070,510. Mixtures of such polishing agents may also be used. The abrasive or polishing agent should not excessively abrade tooth enamel or dentin.

Silica abrasive agents may be particularly preferred for use in the present invention.

Where a formulation prepared according to the invention takes the form of a mouthwash or dentifrice, it may for example contain a water/alcohol (e.g. water/ethyl alcohol) solution and optionally one or more other ingredients selected for example from flavourings, sweeteners, humectants, surfactants, emulsifiers if necessary and mixtures thereof. Suitable humectants include those described above, in particular glycerol and sorbitol. One or more additional antimicrobial agents may also be included.

Non-soap surfactants (for example nonionic, cationic or amphoteric surfactants) may be preferred for use in mouthwash formulations. Suitable nonionic surfactants include the condensation products of hydrophilic alkylene oxide group-containing compounds with organic hydrophobic compounds, as described above. Other suitable nonionic synthetic detergents include: the polyethylene oxide condensates of alkyl phenols; those derived from the condensation of ethylene oxide with the product resulting from the reaction of propylene oxide and ethylene diamine; the condensation products of aliphatic alcohols having from 8 to 18 carbon atoms with ethylene oxide; and the polyoxyethylene derivatives of fatty acid partial esters of sorbitol anhydride (for example the commercially available Tween® products).

Suitable cationic detergents include quaternary ammonium compounds, in particular those having one long alkyl chain of about 8 to 18 carbon atoms, for example lauryl trimethylammonium chloride, cetyl pyridinium chloride, cetyl trimethylammonium bromide, di-isobutylphenoxyethyldimethylbenzylammonium chloride, coconutalkyltrimethylammonium nitrite, cetyl pyridinium fluoride and the like.

Suitable amphoteric detergents include derivatives of aliphatic secondary and tertiary amines in which one of the aliphatic substituents contains from about 8 to 18 carbon atoms and one contains an anionic water solubilising group such as carboxylate, sulphate, sulphonate, phosphate or phosphonate.

Other suitable surfactants, for use in formulations according to the invention, may be found in McCutcheon's Detergents and Emulsifiers and in U.S. Pat. No. 4,051,234.

A formulation prepared according to the invention, when intended for topical application within the oral cavity, may contain one or more agents selected from abrasives, bleaching agents, tooth whitening agents (e.g. peroxides or sodium perborate), surface active agents/detergents as described above, foaming agents, sources of fluoride ions or fluorine-containing ions, zinc salts, non-cariogenic sweeteners such as saccharin or aspartame or dextrose or levulose, other flavourings such as peppermint or spearmint or aniseed, menthol, desensitising agents, anti-tartar/sequestering agents or anti-calculus agents (for example metal salts such as zinc chloride, zinc acetate or zinc oxide; pyrophosphate salts such as alkali metal or ammonium pyrophosphates; or diphosphonates), sodium bicarbonate, anionic polycarboxylates, enzymes such as lactoperoxidases, humectants as described above, binders such as carboxyvinyl polymers, pH regulating buffers, preservatives, colours/dyes (for example chlorophyll or titanium dioxide), plant extracts, anti-plaque agents, additional antimicrobial (for example antifungal or antibacterial, especially antibacterial) agents, and mixtures thereof.

In this context an additional antimicrobial agent may be selected from the group consisting of biocides, disinfectants, antiseptics, antibiotics, bacteriophages, enzymes, anti-adhesins, immunoglobulins and mixtures thereof; it is preferably active as a bactericide, in particular against S. mutans and/or P. gingivalis and/or one or more other bacteria associated with oral health problems.

Again however it may be preferred for the compound (III) or derivative to be the only active agent in the formulation, or at least to be the only antibacterially active agent and/or the only agent active against S. mutans and/or the only agent active against P. gingivalis.

Suitable sources of fluoride or fluorine-containing ions are water soluble fluorides such as water soluble alkali metal or alkaline earth metal fluorides, for example sodium, potassium and barium fluorides (in particular alkali metal fluorides); copper fluorides, such as cuprous fluoride; tin fluorides; fluorosilicates such as sodium or ammonium fluorosilicate; fluorozirconates such as sodium or ammonium fluorozirconate; monofluorophosphates such as sodium or potassium monofluorophosphate; mono-, di- and tri-aluminium fluorophosphates; and fluorinated pyrophosphates such as fluorinated sodium calcium pyrophosphate.

It may be necessary to include a solvent or solubilising agent in a formulation prepared according to the invention, in order to solubilise another agent (in particular an active agent) present in the formulation. Suitable solvents/solubilising agents include flavour oils, polyethylene glycols (preferably those having molecular weights of from about 200 to about 600), propylene glycol, dipropylene glycol, methyl cellosolve, ethyl cellosolve, olive oil, castor oil, amyl acetate, ethyl acetate, glyceryl tristearate, benzyl benzoate and mixtures thereof.

In the case where the formulation is intended for application to a tissue surface within or around the vagina, it may for example be in the form of a cream, foam, gel, ointment, spray, douche or pessary, or it may be carried on or in a tampon, sanitary dressing or wipe.

In the case where the formulation is intended for use in the treatment of body odour, it will suitably contain an anti-perspirant such as an aluminium or aluminium-zirconium salt. It may be in the form of an aerosol, or of a roll-on or “stick” deodorant of known type, containing appropriate conventional liquid or solid carriers and excipients. It may contain one or more perfumes.

In the case where the formulation is intended for use in the treatment of an ocular infection, it may take the form of a cream or ointment, or of eye drops, or of an eye rinse. Such formulations are typically aqueous based, and may contain conventional excipients, including thickeners where appropriate.

In the case where the formulation is intended for use in the treatment (which includes controlling the transmission of) a food-borne infection, it may be in the form of a skin wash (for example a hand wash), or of a surface disinfectant such as a spray. It may be carried in or on a cloth, wipe, brush or other cleaning utensil, or a substrate such as a food preparation surface or implement or a food packaging material; in such cases an item may be impregnated with, or coated with, the formulation.

Where the formulation is intended for application to a non-living area or surface, for instance as a disinfectant, it may take the form of a solution or suspension of the compound (III) or derivative in an appropriate fluid vehicle such as an alcohol or a water/alcohol mix. Again conventional excipients and other additives may be included, as may one or more additional antimicrobial (in particular antibacterial) agents.

Generally speaking a formulation prepared according to the invention may contain one or more agents which enhance the activity of another active agent present in the formulation, or reduce a side effect of such an active, or improve patient compliance on administration of the formulation. It may contain one or more agents which facilitate penetration of an active agent, in particular the compound (III) or derivative, into microbial biofilms. It may contain one or more agents which control the site and/or rate of release of an active agent, in particular the compound (III) or derivative, following administration.

In cases it may be preferred for a formulation prepared or used according to the invention not to contain a polymeric material.

In cases it may be preferred for the formulation not to be or contain a thermoplastic resin, for instance of the type disclosed in U.S. Pat. No. 4,891,391. It may be preferred for the formulation not to contain any additional antimicrobial agents of the types listed in columns 1 and 2 of U.S. Pat. No. 4,891,391.

In cases it may be preferred for the formulation not to contain 2,2-dibromo-3-nitrilopropionamide.

In cases it may be preferred for the formulation not to contain a zinc compound, in particular an antivirally active zinc compound such as those disclosed in U.S. Pat. No. 6,855,341.

In cases it may be preferred for the formulation not to contain empenthrin.

A formulation prepared or used according to the invention may be suitable for, more preferably adapted for, use in an area or on a surface other than living tissue, for instance to treat floors or walls (whether internal or external), work surfaces or instruments, to disinfect contact lenses or to cleanse hair or nails so as to reduce microbe levels. It may be suitable for application to growing or harvested crops, foodstuffs, non-living tissue (for instance for use as a preservative) or clothing (for instance for bio-agent decontamination). It may be used in a food product (which includes a beverage, or a food or beverage additive or ingredient), to control the growth of potentially harmful bacteria. In these cases the excipients, vehicles and/or other additives included with the compound (III) or derivative may be different to those included in a topical skin care or oral health care formulation, but again may be conventional as known for use in such contexts.

Where the formulation is intended for application to a non-living area or surface, for instance as a disinfectant, it may contain a higher concentration of the compound (III) or derivative, for example up to 10 or 15 or 20% w/v, such as from 1 or 2 or 5 or 10 to 20% w/v or from 1 or 2 or 5 to 15% w/v.

A formulation prepared or used according to the invention may be incorporated into, and hence applied in the form of, another product such as a cosmetic, a skin or hair care preparation (for example a skin cleanser, toner or moisturiser, or a shampoo, conditioner, styling mousse or gel or hair spray), a deodorant or anti-perspirant, a dental or other oral health care preparation (for example a toothpaste, mouthwash, dentifrice, dental gel or dental floss, in particular a toothpaste or mouthwash), a cleansing preparation (for example a hand wash for use by surgeons prior to treating patients), a pharmaceutical (which includes veterinary) preparation, a cosmeceutical preparation, a toiletry product (for instance a bath or shower additive or a soap), a laundry or other fabric treatment product or an agricultural or horticultural product. An aspect of the present invention can provide such a product. Moreover the invention can embrace the use of a compound (III) or derivative thereof in the manufacture of such a product for use in the treatment of a bacterial condition.

A formulation prepared or used according to the invention may be marketed with an indication that it has antibacterial activity, for example against one or more of the pathogens referred to above. The marketing of such a formulation may for example include an activity selected from (a) enclosing the formulation in a container or package that comprises the relevant indication; (b) packaging the formulation with a package insert that comprises the indication; (c) providing the indication in a publication that describes the formulation; and (d) providing the indication in a commercial which is aired for instance on the radio, television or internet.

The invention may involve assessing the antibacterial activity of the formulation during or after its preparation, for instance against one or more of the pathogens referred to above. It may involve assessing the antibacterial activity of the formulation both before and after incorporation of the compound (III) or derivative, for example so as to confirm that the compound (III) or derivative contributes to the antibacterial activity of the formulation.

A fourth aspect of the present invention provides a method for controlling the growth of a bacterium, the method comprising applying, to an area or surface which is infected or suspected to be infected or capable of becoming infected with the bacterium, a compound of formula (III) or a derivative thereof. The compound (III) or derivative is suitably applied in a formulation of the type described above, preferably topically. It may in particular be applied to an area or surface which is infected with the bacterium.

“Controlling the growth” of a bacterium embraces inhibiting or preventing its growth, whether completely or partially, as well as killing either completely or partially a culture of the bacterium. It also embraces reducing the risk of subsequent growth of the bacterium in or on the area or surface being treated. It may embrace reducing the risk of transmission of the bacterium from the area or surface being treated to another area or surface and/or living body. The method of the invention may thus be used to treat an existing occurrence of the bacterium or to prevent a potential subsequent occurrence. Controlling the growth of a bacterium may also embrace the disruption and/or suppression of biofilm formation by the organism, as described above.

Again the area or surface to which the compound (III) or derivative is applied will typically be a surface such as human or animal tissue, in particular the skin, nares or a tissue surface within the oral cavity, typically of a living human or animal. In this case the compound (III) or derivative may be applied for therapeutic purposes or for non-therapeutic (e.g. purely cosmetic) purposes. Thus the method of the fourth aspect of the invention encompasses a method of treatment of a human or animal patient suffering from or at risk of suffering from a condition which is caused by, transmitted by and/or exacerbated by (in particular either caused or transmitted by) bacterial activity, the method involving administering to the patient a therapeutically (which term includes prophylactically) effective amount of an antibacterial formulation containing a compound of formula (III) or a pharmaceutically acceptable derivative thereof. Again the bacterial condition may be any of those referred to above in connection with the first to the third aspects of the invention. The compound (III) or derivative is suitably administered in an antibacterially effective amount.

The invention also embraces a method of treatment of a human or animal patient suffering from or at risk of suffering from a condition which is caused or exacerbated by (in particular caused by) sebaceous gland secretions, or any other sebaceous gland disorder, the method involving administering to the patient a therapeutically (which term includes prophylactically) effective amount of a formulation containing a compound of formula (III) or a pharmaceutically acceptable derivative thereof. The compound (III) or derivative is suitably administered in a sebaceous gland secretion inhibiting amount. It may be administered orally, transdermally or topically, preferably either orally or topically, more preferably topically.

Alternatively the compound (III) or derivative may be applied to a non-living area or surface such as in a hospital, dental surgery or food preparation area. For example the method of the fourth aspect of the invention may be used to treat work surfaces, surgical or other instruments (including for example dental instruments, and toothbrushes or other personal oral health care implements), surgical implants or prostheses (including dental components such as caps, bridges and filling materials), protective clothing such as surgical gloves, contact lenses, foods, crops, industrial plant, floors or walls (both internal and external), clothing, bedding, furniture and many other surfaces.

According to a fifth aspect, the present invention provides an antibacterial formulation containing a compound of formula (III) or a pharmaceutically acceptable derivative thereof, together with a pharmaceutically acceptable vehicle, the formulation being adapted for topical application, in particular to the human skin or nares and/or to a tissue surface within the oral cavity. The formulation may contain an anti-perspirant.

The invention also provides, according to a sixth aspect, a product which incorporates an antibacterial formulation according to the fifth aspect.

According to a seventh aspect, the invention provides a method of increasing the antibacterial activity of a formulation, by adding to the formulation a compound of formula (III) or derivative thereof. The formulation may for example be a pharmaceutical formulation, typically one which is suitable and/or adapted and/or intended for topical application. The compound (III) or derivative is suitably added in an antibacterially effective amount.

Throughout the description and claims of this specification, the words “comprise” and “contain” and variations of the words, for example “comprising” and “comprises”, mean “including but not limited to”, and do not exclude other moieties, additives, components, integers or steps.

Throughout the description and claims of this specification, the singular encompasses the plural unless the context otherwise requires. In particular, where the indefinite article is used, the specification is to be understood as contemplating plurality as well as singularity, unless the context requires otherwise.

Preferred features of each aspect of the invention may be as described in connection with any of the other aspects.

Other features of the present invention will become apparent from the following examples. Generally speaking the invention extends to any novel one, or any novel combination, of the features disclosed in this specification (including any accompanying claims and drawings). Thus features, integers, characteristics, compounds, chemical moieties or groups described in conjunction with a particular aspect, embodiment or example of the invention are to be understood to be applicable to any other aspect, embodiment or example described herein unless incompatible therewith.

Moreover unless stated otherwise, any feature disclosed herein may be replaced by an alternative feature serving the same or a similar purpose.

The present invention will now be further described with reference to the following non-limiting examples.

### DETAILED DESCRIPTION

Experimental tests were conducted to determine the antibacterial activity of formulations prepared according to the invention.

Test Micro-Organisms

A wide range of test micro-organisms was used, representing a number of different genera and illustrating a range of potential uses for the present invention.

The first two test micro-organisms represented the staphylococci and propionibacteria genera.

The propionibacterial strain used was Propionibacterium acnes NCTC 737. This is the type strain of the genus; it is fully susceptible to antibiotics.

The propionibacteria are clinically significant due to their involvement in acne. This is a very common, complex and multi-factorial skin disease in which P. acnes and other Propionibacterium spp. (for example P. granulosum) play key roles. They are also opportunistic pathogens in compromised hosts. Thus, activity observed against these micro-organisms is expected to be a good predictor of activity against acne.

The staphylococcal strain used was Staphylococcus aureus ATCC 29213. S. aureus and other staphylococci are common causes of a wide range of skin, skin structure and wound infections; S. aureus is also known to exacerbate eczema. The ATCC 29213 strain is known to be susceptible to beta-lactam antibiotics such as methicillin.

Activity observed against these micro-organisms is expected to be a reasonable qualitative predictor of antibacterial activity generally, in particular of activity against micro-organisms responsible for skin and skin structure infections.

The propionibacteria were cultured and maintained on Wilkins-Chalgren Anaerobe Medium (agar and broth) at pH 6.0; all cultures were incubated anaerobically at 37° C. for 72 hours.

The staphylococci were cultured and maintained on Mueller-Hinton Medium (agar and broth) at pH 7.2; cultures were incubated aerobically at 37° C. for 19-20 hours.

Two further test microbial species were used to demonstrate the suitability of compounds of formula (III) for the treatment of conditions affecting the oral cavity. The first was Porphyromonas gingivalis. This is a black pigmented Gram-negative anaerobic bacterium belonging to the genus Porphyromonas. P. gingivalis is an oral pathogen typically associated with periodontal lesions, infections and adult periodontal disease. Gingivitis (inflammation of the gums that causes bleeding and exposes the base of the teeth) can be a precursor to periodontal disease by allowing P. gingivalis to infect the areas near the roots of the teeth and thus to cause tooth decay and infection.

Activity observed against this micro-organism is expected to be a reasonable qualitative predictor of antibacterial activity against micro-organisms responsible for periodontal lesions and infections and periodontal disease. Tests were performed with three different strains of P. gingivalis, namely NCTC 11834, ATCC BAA-308 and ATCC 53978.

Also tested was Streptococcus mutans—this is a Gram-positive, microaerophilic bacterium associated principally with the human oral cavity. Clinically, S. mutans plays a significant role in dental caries and in infective endocarditis. The bacterium produces lactic acid as a by-product of its normal metabolism, and also produces an enzyme (dextransucrase) that can utilise sucrose to produce an extracellular dextran-based polysaccharide. This polysaccharide enables the bacteria to adhere to each other on the surface of a tooth to form plaque. It is the combination of the plaque and lactic acid that can result in tooth decay. More seriously, if the bacterium enters the bloodstream, for example after a tooth extraction, it can bind to the endocardium within the heart and if left untreated can prove fatal.

Activity observed against this micro-organism is thus expected to be a reasonable qualitative predictor of antibacterial activity against micro-organisms responsible for dental caries and infective endocarditis. Tests were performed on two different strains of S. mutans, namely ATCC 25175 and ATCC 35668. Tests were also performed on S. sobrinus ATCC 33478 (see below), another bacterium implicated in dental caries.

The P. gingivalis strains were cultured and maintained on Wilkins-Chalgren Anaerobe Medium (agar and broth) at pH 7.0; all cultures were incubated anaerobically at 37° C. for 5-7 days.

The S. mutans strains were cultured and maintained on Wilkins-Chalgren Anaerobe Medium (agar and broth) at pH 7.0 supplemented with 1 g/L glucose; all cultures were incubated at 37° C. for 48 h in an atmosphere containing 5% CO₂.

The following additional test organisms were also used:

| | | Culture conditions | | | |
|---|---|---|---|---|---|
| | | Growth | Temp | | |
| Test organism | Gram test | media | (° C.) | Atmosphere | Time (h) |
| Actinomyces naeslundii | + | WC pH7 | 37 | 5% CO₂ | 24 |
| ATCC 12104 | | | | | |
| Bacillus cereus ATCC | + | MH | 30 | Aerobic | 24 |
| 11778 | | | | | |
| Clostridium difficile ATCC | + | WC pH7* | 37 | Anaerobic | 48 |
| 7000057 | | | | | |
| Clostridium sporogenes | + | WC pH7 | 37 | Anaerobic | 24 |
| ATCC 3584 | | | | | |
| Corynebacterium | + | MH | 37 | Aerobic | 24 |
| mucifaciens ATCC 700355 | | | | | |
| Enterococcus faecalis ATCC | + | MH | 37 | Aerobic | 24 |
| 29212 | | | | | |
| Gardnerella vaginalis | − | MH + 5% | 37 | 5% CO₂ | 48 |
| ATCC 14018 | | lysed | | | |
| | | blood | | | |
| | | (broth) | | | |
| | | WC pH7 | | | |
| | | (agar) | | | |
| Listeria monocytogenes | + | MH | 37 | Aerobic | 24 |
| ATCC 15313 | | | | | |
| Prevotella nigrescens ATCC | − | WC pH7 | 37 | Anaerobic | 48 |
| 33563 | | | | | |
| Streptococcus pyogenes | + | MH | 37 | 5% CO₂ | 48 |
| ATCC 12344 | | | | | |
| Streptococcus sobrinus | + | WC pH7 + | 37 | 5% CO₂ | 48 |
| ATCC 33478 | | 1 g/L | | | |
| | | glucose | | | |

[Abbreviations: American Type Culture Collection (ATCC), Mueller-Hinton (MH), Wilkins-Chalgren (WC), *denotes where agar medium was needed to maintain the organism 5% fresh horse blood was added to the media]

Of the above pathogens, Bacillus cereus is associated with food poisoning; Enterococcus faecalis and Listeria monocytogenes are both food borne pathogens, the former being implicated in burn and wound infections. Clostridium sporogenes is associated with food spoilage, whilst Clostridium difficile may also be spread by contaminated food.

Corynebacterium mucifaciens is closely related to organisms (aerobic diphtheroids of the genus Corynebacterium) that cause body odour. Prevotella nigrescens and Actinomyces naeslundii are implicated in periodontitis and other anaerobic infections, and Streptococcus sobrinus in dental caries. Streptococcus pyogenes is associated with skin and soft tissue infections. Gardnerella vaginalis can cause vaginitis, vaginosis and certain sexually transmitted infections.

The following tests were carried out to assess antibacterial activity against the test organisms.

(a) Minimum Inhibitory Concentration (MIC) Assay

This is a standard international method for quantitatively assessing the antimicrobial activity of a compound in a liquid medium. The method used a sterile 96-well microtitre plate, capable of holding about 200 μl of liquid per well. The wells contained liquid culture medium and ranges of decreasing concentrations of the relevant test compound in doubling dilutions (e.g. 1000, 500, 250, 125 . . . μg/ml, etc. down to 0.49 μg/ml). The culture media were as described above.

The wells were inoculated with a liquid suspension of freshly grown micro-organism and incubated under the conditions described above. After incubation, the microtitre plate was examined visually (with the aid of a light box) for cloudiness in each well, which would indicate microbial growth. The MIC value was recorded as the lowest concentration of test compound required to inhibit microbial growth, i.e. the lowest concentration for which the liquid in the well remained clear.

The assays included both negative (culture medium with no micro-organisms) and positive (culture medium plus diluting solvent plus micro-organism) controls.

Since inhibition does not necessarily indicate killing of microbial cells, merely that growth as visible to the naked eye has been inhibited, it is desirable to conduct a further test (the MBC assay described below) to establish the concentration of the test compound needed to kill the test organism.

(b) Minimum Biocidal (Bactericidal) Concentration (MBC) Assay

This assay, normally carried out after an MIC assay, determines the minimum concentration of a compound that is lethal to the micro-organism being tested.

Following an MIC assay, a 5 μl sample was withdrawn from the first microtitre well that showed positive growth and from all the subsequent wells that showed no growth. These samples were then individually sub-cultured on antibiotic-free agar medium, under the incubation conditions described above. Following incubation they were examined visually for microbial growth. The MBC was taken to be the lowest test compound concentration for which the incubated sample showed no growth.

The ratio of MIC to MBC should ideally be as close to 1 as possible. This facilitates selection of the lowest possible effective concentration of a test compound with a reduced risk of selecting a sub-lethal concentration which could promote resistance or allow the target microbial population to recover.

(c) Agar Dilution MIC Assay

This is a standard international method for quantitatively assessing the antimicrobial activity of a compound in a solid medium. The test compound was prepared to 40× the highest concentration required (e.g. 10 mg/ml for a final concentration of 250 μg/ml) and a series of doubling dilutions were performed in a suitable solvent. A set amount of these antimicrobial stock solutions was then added to molten agar medium (ca. 55° C.), mixed thoroughly, poured into sterile Petri dishes and allowed to cool/set. The culture medium was as described above.

A Multipoint™ Inoculator (AQS Manufacturing Ltd, UK) was used to inoculate the plates by spotting the inocula onto the surface of the agar, delivering approximately 1 to 2 μl per spot (yielding 10⁵ CFU (colony forming units) per spot).

The plate(s) were then incubated under the conditions described above, following which they were examined visually for signs of bacterial growth. The MIC value was ascertained when there was a marked reduction in, or total loss of, growth on the test plate at the lowest concentration as compared to that of the growth on the control plate.

The assays were conducted in triplicate and included a positive control (culture medium, diluting solvent and inoculum).

### Example 1

### Activity Against Propionibacterium spp—AO 2246

These experiments used P. acnes NCTC 737 as the test organism.

MIC and MBC assays, as described above, were carried out using as the test compound AO 2246 (ex Sigma Aldrich, UK), dissolved in DMSO. All the experiments were conducted in triplicate.

The results are shown in Table 1 below; all are collated from a number of experiments.

TABLE 1

| | |
|---|---|
| MIC (μg/ml) | 3.9 |
| MBC (μg/ml) | 15.6 |
| MIC/MBC ratio | 0.25 |

It can be seen from Table 1 that the AO 2246 is highly active as an antibacterial agent against P. acnes NCTC 737. This indicates its likely activity as an anti-acne agent, the propionibacteria being implicated in acne.

### Example 2

### Activity Against Staphylococci—AO 2246

These experiments used S. aureus ATCC 29213 as the test organism.

MIC and MBC assays, as described above, were carried out using AO 2246 as the test compound, again dissolved in DMSO. All the experiments were conducted in triplicate.

The results are shown in Table 2 below; all are collated from a number of experiments.

TABLE 2

| | |
|---|---|
| MIC (μg/ml) | 3.9 |
| MBC (μg/ml) | 7.8 |
| MIC/MBC ratio | 0.5 |

It can be seen from Table 2 that the AO 2246 is highly active as an antibacterial agent against S. aureus ATCC 29213. These data indicate the likely utility of the compound in the prevention and/or treatment of staphylococcal infections such as MRSA.

### Example 3

### Activity Against Propionibacterium spp—AO 425

Example 1 was repeated, using as the test compound AO 425 (ex Sigma Aldrich, UK), dissolved in DMSO. All the experiments were conducted in triplicate.

The results are shown in Table 3 below; all are collated from a number of experiments.

TABLE 3

| | |
|---|---|
| MIC (μg/ml) | 7.8 |
| MBC (μg/ml) | 31.25 |
| MIC/MBC ratio | 0.25 |

It can be seen from Table 3 that the AO 425 is also highly active as an antibacterial agent against P. acnes NCTC 737. This indicates its likely activity as an anti-acne agent.

In contrast, other structurally similar alkyl-bis-phenol compounds were found to be far less active against this test organism than AO 2246 and AO 425. 2,2′-ethylidene-bis(4,6-di-tert-butylphenol) and 4,4′-methylenebis(2,6-di-tert-butylphenol) for example, when tested against P. acnes NCTC 737, were both found to have MIC and MBC values greater than 250 μg/ml.

### Example 4

### Activity Against Staphylococci—AO 425

Example 2 was repeated, again using AO 425 dissolved in DMSO. All the experiments were conducted in triplicate.

The results are shown in Table 4 below; all are collated from a number of experiments.

TABLE 4

| | |
|---|---|
| MIC (μg/ml) | 7.8 |
| MBC (μg/ml) | 62.5 |
| MIC/MBC ratio | 0.125 |

The Table 4 data again show that the AO 425 is highly active as an antibacterial agent against S. aureus ATCC 29213, and indicate the likely utility of the compound in the prevention and/or treatment of staphylococcal infections such as MRSA.

Again, other structurally similar alkyl-bis-phenol compounds were found to be far less active against this test organism than AO 2246 and AO 425. 2,2′-ethylidene-bis(4,6-di-tert-butylphenol) and 4,4′-methylenebis(2,6-di-tert-butylphenol) both had MIC and MBC values greater than 250 μg/ml against S. aureus ATCC 29213.

### Example 5

### Activity Against Other Propionibacterium spp

The activity (MIC by agar dilution) of AO 2246 was determined against a panel of different propionibacterium strains. DMSO was used as the solvent. The MIC experiments were performed in triplicate.

The results are shown in Table 5 below, which also indicates the resistance phenotype for each of the test species/strains.

TABLE 5

| | Resistance | MIC |
|---|---|---|
| Test organism | Phenotype | (μg/ml) |
| Propionibacterium acnes NCTC 737 | None | 3.9 |
| Propionibacterium granulosum NCTC 11865 | None | 1.95 |
| P. acnes PRP-002 | Tet/MLS | 3.9 |
| P. acnes PRP-003 | Tet | 3.9 |
| P. acnes PRP-004 | Tet | 3.9 |
| P. granulosum PRP-005 | MLSK | 3.9 |
| P. granulosum PPR-006 | MLS | 1.95 |
| P. acnes PPR-007 | Clin | 1.95 |
| P. acnes PRP-008 | Clin | 1.95 |
| P. acnes PRP-010 | MLSK | 3.9 |
| P. acnes PRP-017 | MLS | 3.9 |
| P. granulosum PRP-019 | MLSK | 3.9 |
| P. granulosum PRP-021 | MLS | 3.9 |
| P. acnes PRP-023 | MLSK | 3.9 |
| P. acnes PRP-026 | MLS | 3.9 |
| P. acnes PRP-039 | Tet/MLS | 3.9 |
| P. granulosum PRP-043 | MLS | 1.95 |
| P. granulosum PRP-044 | MLS | 3.9 |
| P. acnes PRP-046 | None | 3.9 |
| P. acnes PRP-053 | Tet/MLS | 3.9 |
| P. granulosum PRP-055 | None | 3.9 |
| P. acnes PRP-059 | MLS | 3.9 |
| P. acnes PRP-068 | Ery | 3.9 |
| P. acnes PRP-101 | Tet/MLS | 3.9 |
| P. acnes PRP-102 | Tet/MLS | 3.9 |

[Abbreviations: American Type Culture Collection (ATCC), National Collection of Type Cultures (NCTC), Propionibacterium Panel Number (PRP), Tetracycline (Tet), Erythromycin (Ery), Clindamycin (Clin), Macrolide-Lincosamide-Streptogramin (MLS), Macroliode-Lincosamide-Streptogramin-Ketolide (MLSK).]

AO 2246 can be seen to possess an excellent level of activity against the wide range of propionibacterium strains tested.

### Example 6

### Activity Against Other Staphylococci

The activity (MIC and MBC) of AO 2246 was determined against a panel of different staphylococcal strains. DMSO was used as the solvent. MIC and MBC experiments were performed in triplicate.

The results are shown in Table 6 below, which also indicates the resistance phenotype for each of the test species/strains.

TABLE 6

| | | | | MIC/ |
|---|---|---|---|---|
| | Resistance | MIC | MBC | MBC |
| Test organism | phenotype | (μg/ml) | (μg/ml) | Ratio |
| Staphylococcus simulans | ND | 15.6 | 31.25 | 0.5 |
| ATCC 27848 | | | | |
| Staphylococcus xylosus | ND | 3.9 | 3.9 | 1 |
| ATCC 29971 | | | | |
| Staphylococcus cohnii | ND | 3.9 | 3.9 | 1 |
| ATCC 29974 | | | | |
| Staphylococcus haemolyticus | ND | 3.9 | 3.9 | 1 |
| ATCC 29970 | | | | |
| Staphylococcus warneri | ND | 15.6 | >31.25 | <0.5 |
| ATCC 27836 | | | | |
| Staphylococcus capitis | ND | 3.9 | 15.6 | 0.25 |
| ATCC 27840 | | | | |
| Staphylococcus hominis | ND | 7.8 | 31.25 | 0.25 |
| ATCC 27844 | | | | |
| Staphylococcus auricularis | ND | 1.95 | 3.9 | 0.5 |
| ATCC 33753 | | | | |
| Staphylococcus aureus | ND | 0.98 | 15.6 | 0.06 |
| ATCC 12600 | | | | |
| S. aureus | ND | 0.98 | 15.6 | 0.06 |
| ATCC 12600-U | | | | |
| S. aureus ATCC 12601 | ND | 1.95 | 1.95 | 1 |
| S. aureus ATCC 12602 | ND | 1.95 | 3.9 | 0.5 |
| S. aureus ATCC 12604 | ND | 1.95 | 3.9 | 0.5 |
| S. aureus ATCC 12605 | ND | 1.95 | 1.95 | 1 |
| S. aureus ATCC 12606 | ND | 1.95 | 3.9 | 0.5 |
| S. aureus ATCC 12607 | ND | 1.95 | 7.8 | 0.25 |
| S. aureus ATCC 29213 | ND | 3.9 | 7.8 | 0.5 |
| S. aureus ATCC 25923 | ND | 7.8 | 7.8 | 1 |
| S. aureus | Met/β-lactams* | 1.95 | 7.8 | 0.25 |
| CPHL EMRSA 15 | | | | |
| S. aureus | Met/β-lactams* | 1.95 | 7.8 | 0.25 |
| CPHL EMRSA 16 | | | | |
| S. aureus | Met/β-lactams* | 1.95 | 7.8 | 0.25 |
| CPHL EMRSA 17 | | | | |
| S. aureus | Van* | 1.95 | 7.8 | 0.25 |
| CPHL VISA Mu3 | (intermediate) | | | |
| S. aureus | Van* | 0.98 | 15.6 | 0.06 |
| CPHL VISA Mu50 | (intermediate) | | | |
| S. aureus | Van/Tec* | 1.95 | 3.9 | 0.5 |
| CPHL GISA HO41340156 | (intermediate) | | | |
| S. saprophyticus | ND | 3.9 | 3.9 | 1 |
| NCTC 7292 | | | | |
| S. epidermidis | ND | 3.9 | 7.8 | 0.5 |
| NCTC 11047 | | | | |
| S. aureus NCTC 12981 | ND | 1.95 | 7.8 | 0.25 |
| S. aureus NCTC 12493 | Met* | 0.98 | 1.95 | 0.5 |

[Abbreviations: American Type Culture Collection (ATCC), Central Public Health Laboratory UK (CPHL), National Collection of Type Cultures (NCTC), Methicillin (Met), Vancomycin (Van), Teicoplanin (Tec), not determined (ND), epidemic methicillin resistant S. aureus (EMRSA), vancomycin intermediate S. aureus (VISA), gylcopeptide resistant S. aureus (GISA).]
*Other uncharacterised antibiotic resistances may be present.

Again AO 2246 appears to demonstrate excellent activity against the staphylococcal strains tested.

### Example 7

### Activity Against Other Micro-Organisms—AO 2246

The activity (MIC and MBC) of AO 2246 was determined against a panel of different test micro-organisms. These included S. mutans, P. gingivalis and a number of other bacteria implicated in oral health problems, as well as C. difficile, E. faecalis and L. monocytogenes (a listeria strain). They also included Corynebacterium mucifaciens, a bacterial strain which is closely related to the aerobic diphtheroids implicated in body odour.

DMSO was used as the solvent. MIC and MBC experiments were performed in triplicate.

The results are shown in Table 7 below.

TABLE 7

| | | | MIC/ |
|---|---|---|---|
| | MIC | MBC | MBC |
| Test organism | (μg/ml) | (μg/ml) | Ratio |
| Actinomyces naeslundii ATCC 12104 | 1.95 | 3.9 | 0.5 |
| Bacillus cereus ATCC 11778 | 1.95 | 3.9 | 0.5 |
| Clostridium difficile ATCC 7000057 | 3.9 | 3.9 | 1 |
| Clostridium sporogenes ATCC 3584 | 3.9 | 31.25 | 0.125 |
| Corynebacterium mucifaciens ATCC 700355 | 1.95 | 3.9 | 0.5 |
| Enterococcus faecalis ATCC 29212 | 1.95 | 7.8 | 0.25 |
| Gardnerella vaginalis ATCC 14018 | 15.6 | 15.6 | 1 |
| Listeria monocytogenes ATCC 15313 | 1.95 | 7.8 | 0.25 |
| Porphyromonas gingivalis NCTC 11834 | 3.9 | 3.9 | 1 |
| Porphyromonas gingivalis ATCC BAA-308 | 3.9 | 3.9 | 1 |
| Porphyromonas gingivalis ATCC 53978 | 0.98 | 1.95 | 0.5 |
| Prevotella nigrescens ATCC 33563 | 7.8 | >15.6 | <0.5 |
| Streptococcus mutans ATCC 25175 | 15.6 | 62.5 | 0.5 |
| Streptococcus mutans ATCC 35668 | 7.8 | 31.25 | 0.25 |
| Streptococcus pyogenes ATCC 12344 | 0.98 | 0.98 | 1 |
| Streptococcus sobrinus ATCC 33478 | 15.6 | 31.25 | 0.5 |

[Abbreviations: American Type Culture Collection (ATCC), National Collection of Type Cultures (NCTC).]

The Table 7 data indicate wide ranging antibacterial activity for the AO 2246, in particular against Gram-positive bacteria but also against certain Gram-negative bacteria associated with periodontal diseases.

### Example 8

### Activity Against Other Micro-Organisms—AO 425

Example 7 was repeated, against a similar range of test organisms, using AO 425 as the test compound. Again DMSO was used as the solvent, and both MIC and MBC experiments were performed in triplicate.

The results are shown in Table 8 below.

TABLE 8

| | | | MIC/ |
|---|---|---|---|
| | MIC | MBC | MBC |
| Test organism | (μg/ml) | (μg/ml) | ratio |
| Actinomyces naeslundii ATCC 12104 | 0.98 | 3.9 | 0.25 |
| Bacillus cereus ATCC 11778 | 1.95 | 3.9 | 0.5 |
| Clostridium difficile ATCC 7000057 | 7.8 | 7.8 | 1 |
| Clostridium sporogenes ATCC 3584 | 3.9 | 7.8 | 0.5 |
| Gardnerella vaginalis ATCC 14018 | 15.6 | 31.25 | 0.5 |
| Listeria monocytogenes ATCC 15313 | 0.98 | 7.8 | 0.125 |
| Porphyromonas gingivalis NCTC 11834 | 1.95 | 1.95 | 1 |
| Porphyromonas gingivalis ATCC BAA-308 | 1.95 | 3.9 | 0.5 |
| Porphyromonas gingivalis ATCC 53978 | 0.98 | 0.98 | 1 |
| Prevotella nigrescens ATCC 33563 | 15.6 | >15.6 | <1 |
| Streptococcus mutans ATCC 25175 | 15.6 | 62.5 | 0.25 |
| Streptococcus mutans ATCC 35668 | 7.8 | >250 | <0.03 |
| Streptococcus sobrinus ATCC 33478 | 31.25 | 62.5 | 0.5 |

[Abbreviations: American Type Culture Collection (ATCC), National Collection of Type Cultures (NCTC).]

The Table 8 data indicate wide ranging antibacterial activity for the AO 425. Most Gram-positive micro-organisms were found to be susceptible, as were certain Gram-negative bacteria associated with periodontal disease.

When tested against C. mucifaciens ATCC 700355, E. faecalis ATCC 29212 and S. pyogenes ATCC 12344, AO 425 was found to have significantly lower activity than AO 2246. Thus, it may be preferable to use AO 2246 in the treatment of conditions in which such bacteria are implicated.

### Example 9

### Topical Anti-Acne Formulations

The results from Examples 1, 3 and 5 show that AO 425 and in particular AO 2246 can be effective antibacterial agents, in particular against the bacteria associated with acne. This can be of use in preparing antibacterial formulations, in particular for topical application to the skin, for prophylactic or therapeutic use in any context where such bacteria are thought to be involved as possible sources of infection. More specifically, it can be of use in preparing anti-acne formulations, again suitably for topical use.

A topical formulation for use in treating acne may for example be prepared by formulating a compound of formula (III), such as AO 2246 or AO 425, or a pharmaceutically acceptable derivative thereof, in a suitable fluid vehicle and optionally together with conventional additives. Such vehicles and additives may be for instance as found in Williams' “Transdermal and Topical Drug Delivery”, Pharmaceutical Press, 2003 and other similar reference books, and/or in Rolland A et al, “Site-specific drug delivery to pilosebaceous structures using polymeric microspheres”, Pharm. Res. 1993; 10: 1738-44; Mordon S et al, “Site-specific methylene blue delivery to pilosebaceous structures using highly porous nylon microspheres: an experimental evaluation”, Lasers Surg. Med. 2003; 33: 119-25; and Alvarez-Roman R et al, “Skin penetration and distribution of polymeric nanoparticles”, J. Controlled Release 2004; 99: 53-62.

The formulation may be prepared and administered using known techniques. It may for example take the form of a cream, lotion, foam, ointment or gel.

The concentration of the compound (III) or derivative may be in the ranges described above, and will be determined based on its antibacterial activity and the intended use of the formulation.

### Example 10

### Topical Anti-Staphylococcal Formulations

The results from Examples 2, 4 and 6 show that AO 425 and in particular AO 2246 can be effective anti-staphylococci agents, including against antibiotic resistant strains such as MRSA. This can be of use in preparing antibacterial formulations, in particular for topical application to the skin, for prophylactic or therapeutic use in any context where such bacteria are thought to be involved as possible sources of infection, for example in the nares or ears or indeed on the hands.

A formulation for use against staphylococci such as S. aureus may be prepared by formulating a compound of formula (III), such as AO 2246 or AO 425, or a (preferably pharmaceutically acceptable) derivative thereof, in a similar manner to that described for the anti-acne formulation of Example 9. The ingredients may in this case be formulated as a spray, for instance for application to work surfaces or surgical instruments; as a cleansing gel or lotion for instance for hand washing; as a nasal spray for application to the anterior nares; as drops for use in the ears or eyes; or in many other appropriate forms. Such a formulation may in particular be used prophylactically, e.g. to reduce the risk of outbreaks of MRSA or similar infections.

### Example 11

### Topical Oral Health Care Formulations

Examples 7 and 8 above show that a compound of formula (III) can be active against both S. mutans and P. gingivalis strains, as well as against other bacteria—in particular Actinomycetes naeslundii, Streptococcus sobrinus and Prevotella nigrescens—associated with oral health problems. This indicates the utility of a compound of formula (III) in treating infections caused by the relevant test organisms, in particular infections within the oral cavity such as for example plaque formation, gingivitis, periodontitis or dental caries. Such active agents may also be used for general oral health care, for example for the creation and/or maintenance of fresh-smelling breath. They may also be used to treat more systemic conditions associated with S. mutans or P. gingivalis infections, for example infective endocarditis or cardiovascular diseases.

A topical formulation for use in this way, against S. mutans and/or P. gingivalis, may be prepared by formulating a compound of formula (III), such as AO 2246 or AO 425, or a pharmaceutically acceptable derivative thereof, in a suitable fluid vehicle and optionally together with conventional additives, as described above.

The formulation may be prepared and administered using known techniques. For topical application it may for example take the form of a paste, cream, gel, lozenge, buccal patch, spray, mouthwash or dentifrice, or it may be carried in or on a dental fibre or tape. It may contain additives which target the active ingredient to a particular site, for example the gums or teeth or the sub-gingival regions, and/or which otherwise control the release of the active at the relevant site.

1-53. (canceled)

54. A method for controlling the growth of a bacterium, the method comprising applying, to an area or surface which is infected or suspected to be infected or capable of becoming infected with the bacterium, a compound of formula (III) below: 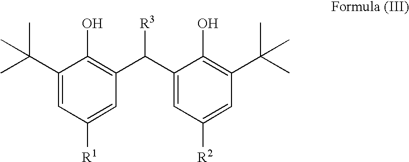
wherein each of R¹ and R² is independently selected from methyl and ethyl and R³ is hydrogen, or a derivative thereof.

55. A method according to claim 54, wherein the bacterium is selected from staphylococci (in particular S. aureus), members of the genus Enterococcus (in particular E. faecalis) and members of the genus Clostridium (in particular C. difficile).

56. A method according to claim 54, wherein the compound (III) or derivative is applied to a non-living area or surface.

57. A method according to claim 54, wherein the compound of formula (III) is Antioxidant 2246.

58. A method of treatment of a human or animal patient suffering from or at risk of suffering from a condition which is caused by, transmitted by and/or exacerbated by bacterial activity, the method involving administering to the patient a therapeutically (which term includes prophylactically) effective amount of an antibacterial formulation containing a compound of formula (III) below: 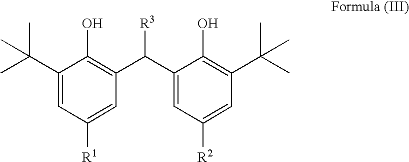
wherein each of R¹ and R² is independently selected from methyl and ethyl and R³ is hydrogen, or a pharmaceutically acceptable derivative thereof.

59. A method according to claim 58, wherein the compound (III) or derivative is administered topically.

60. A method according to claim 58, wherein the compound of formula (III) is Antioxidant 2246.

61. An antibacterial formulation containing a compound of formula (III), as defined in claim 54, or a pharmaceutically acceptable derivative thereof, together with a pharmaceutically acceptable vehicle, the formulation being adapted for topical application and additionally containing an anti-perspirant.

62. A method according to claim 58, wherein the condition is a skin or skin structure condition.

63. A method according to claim 58, wherein the condition is acne or acne lesions.

64. A method according to claim 58, wherein the condition is body odor.

65. A method according to claim 58, wherein the condition is a bacterial condition within the oral cavity.

66. A method according to claim 58, wherein the condition is an ocular condition.
